(19) United States
(12) Patent Application Publication
(10) Pub. No.: US 2022/0412586 A1
(43) Pub. Date: Dec. 29, 2022

(54) DIFFUSER AIR PURIFICATION UNIT

(71) Applicant: Price Industries, Inc., Suwanee, GA (US)

(72) Inventors: Nolan Hosking, Alpharetta, GA (US); Mark Mahon, Winnipeg (CA); Chris Hildebrand, Winnipeg (CA); Tim Ko, Atlanta, GA (US); Michael Holliday, Auburn, GA (US)

(21) Appl. No.: 17/846,256

(22) Filed: Jun. 22, 2022

Related U.S. Application Data
(60) Provisional application No. 63/213,966, filed on Jun. 23, 2021.

(51) Int. Cl. F24F 8/20; A61L 9/20; A61L 9/22; F24F 8/108; F24F 8/30; F24F 8/90; F24F 11/30

(52) U.S. Cl. CPC F24F 8/20; A61L 9/20; A61L 9/22; F24F 8/108; F24F 8/30; F24F 8/90; F24F 11/30; A61L 2209/111; A61L 2209/134; A61L 2209/14; F24F 2120/10

(57) ABSTRACT

Disclosed herein is a diffuser air purification unit. The diffuser air purification unit can comprise a diffuser, a filtration chamber, a purification chamber, a blower, a housing, and a control module. The filtration chamber can be configured to receive air through the diffuser from an external setting in which the diffuser air purification unit is installed and to cause a filtration of the air when the air flows through the filtration chamber. The purification chamber can be configured to receive the air from the filtration chamber and to cause a purification of the air when the air flows through the purification chamber. The blower can be configured to intake the air from the external setting through the diffuser, the filtration chamber, and the purification chamber and to expel the air into the external setting. The housing can at least partially enclose the filtration chamber and the purification chamber. The control module can be secured to the housing and be configured to control an operation of the diffuser air purification unit.

### CROSS-REFERENCE TO RELATED APPLICATIONS

This application claims the benefit of and priority to co-pending U.S. Provisional App. No. 63/213,966, entitled “DIFFUSER AIR PURIFICATION UNIT,” and filed on Jun. 23, 2021, which is incorporated by reference herein in its entirety.

### BACKGROUND

In indoor settings like offices, circulated air can include dust, airborne pathogens, and other airborne particles that have a negative effect on health. Thus, steps need to be taken to ensure that the air is clean and safe. Ceiling air diffusers that are installed in many buildings can help direct air flow to or from a room, but they cannot fully purify and clean the air. At best, ceiling diffusers might include ultraviolet light air sanitizers, but they do not include filtration.

Yet adding filtration to existing diffusers can disrupt the operation of a building's airflow system. For example, a filter would change or interrupt airflow in a way that the airflow system may not be designed. This would occur because of the pressure needed to get air to pass through the filter. Thus, costly refits to the airflow system would need to be performed to allow filters to be added to diffusers. Additionally, these refits would still likely achieve only low efficiency filtration. Integrated high efficiency filtration is typically achieved using extensive infrastructure that makes such filtration practical only in critical settings like operating rooms.

High-efficiency filtration can be achieved using portable air filtration units. However, these units are conspicuous and can consume a considerable amount of space in smaller settings. Furthermore, these units may not include air purification mechanisms like ultraviolet light or bipolar ionization.

### BRIEF DESCRIPTION OF THE DRAWINGS

For a more complete understanding of the embodiments and the advantages thereof, reference is now made to the following description, in conjunction with the accompanying figures briefly described as follows:

FIGS. 1A-B illustrate examples of perspective views of the diffuser air purification unit, according to various embodiments of the present disclosure.

FIGS. 2A-B illustrate examples of top views of the diffuser air purification unit, according to various embodiments of the present disclosure.

FIGS. 3A-C illustrate examples of bottom views of the diffuser air purification unit, according to various embodiments of the present disclosure.

FIGS. 4A-B illustrate examples of first side views of the diffuser air purification unit, according to various embodiments of the present disclosure.

FIGS. 5A-B illustrate examples of second side views of the diffuser air purification unit, according to various embodiments of the present disclosure.

FIG. 6 illustrates an example of an isolated perspective view of a control module of the diffuser air purification unit, according to various embodiments of the present disclosure.

FIG. 7 illustrates an example of a flow diagram of a process for purifying air using the diffuser air purification unit, according to various embodiments of the present disclosure.

The drawings illustrate only example embodiments and are therefore not to be considered limiting of the scope described herein, as other equally effective embodiments are within the scope and spirit of this disclosure. The elements and features shown in the drawings are not necessarily drawn to scale, emphasis instead being placed upon clearly illustrating the principles of the embodiments. Additionally, certain dimensions may be exaggerated to help visually convey certain principles. In the drawings, similar reference numerals between figures designate like or corresponding, but not necessarily the same, elements.

### DETAILED DESCRIPTION

In the following paragraphs, the embodiments are described in further detail by way of example with reference to the attached drawings. In the description, well known components, methods, and/or processing techniques are omitted or briefly described so as not to obscure the embodiments. As used herein, the “present disclosure” refers to any one of the embodiments described herein and any equivalents. Furthermore, reference to various feature(s) of the “present embodiment” is not to suggest that all embodiments must include the referenced feature(s).

Disclosed herein is a diffuser air purification unit that can filter and purify air with the versatility of standard diffusers and the efficiency of portable air filtration units. The diffuser air purification unit can filter air at a higher efficiency than filtration solutions in typical settings. For example, the diffuser air purification unit can use high efficiency particulate air (HEPA) filtration to continually clean the air. The diffuser air purification unit can substantially increase the quantity of air changes in a setting relative to typical settings. The diffuser air purification unit can even surpass the standards set by The American Society of Heating, Refrigerating and Air-Conditioning Engineers (ASHRAE) for number of HEPA-filtered air changes per hour. In addition, the diffuser air purification can further treat the filtered air using, for example, ultraviolet light and bipolar ionization.

The diffuser air purification unit can be a standalone unit that does not need to be connected to a building's heating, ventilation, and air conditioning (HVAC) ductwork. Likewise, the diffuser air purification unit can be self-contained in a package that can be easily incorporated into existing indoor settings. While buildings are typically not built to support high efficiency filtration, existing buildings can be retrofitted to include the diffuser air purification unit. The diffuser air purification unit can be constructed so that all its components fit within a standard cell of a suspension grid drop ceiling. Likewise, the diffuser air purification unit can be constructed so that it fits universally in existing buildings with different ceiling heights.

The diffuser air purification unit can include different types of diffusers to match with those already present in a setting. The diffuser air purification unit can be installed or retrofitted in existing buildings in rooms or areas where air purification is desired. This allows the flexibility to select settings in which to install the diffuser air purification unit based on each setting's respective air quality requirements. The diffuser air purification unit can provide access to internal components on multiple sides and from the diffuser for maintenance and service. In some examples, maintenance can be performed on the diffuser air purification unit without using tools.

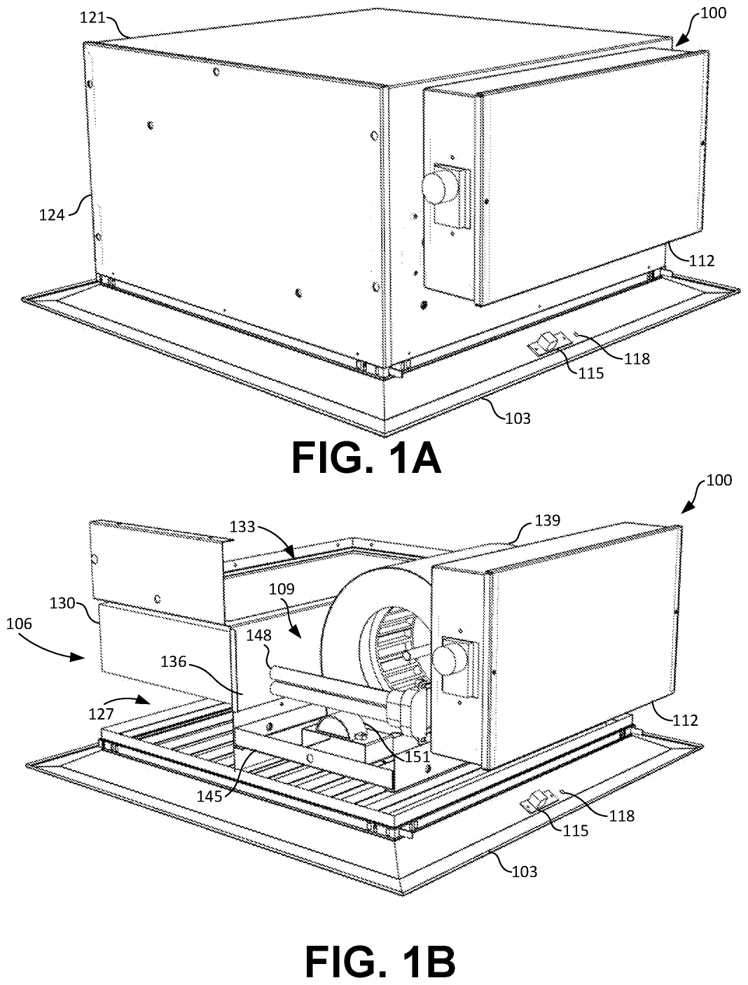

FIGS. 1A-B illustrate examples of perspective views of the diffuser air purification unit 100. FIG. 1A shows an outside of the diffuser air purification unit 100, while FIG. 1B shows the internal components of the diffuser air purification unit 100. The diffuser air purification unit 100 can include a diffuser 103, a filtration chamber 106, a purification chamber 109, a control module 112, an occupancy sensor 115, and a status indicator 118. The filtration chamber 106 and the purification chamber 109 can be enclosed within a housing 121 and access panels 124.

In some examples, the diffuser air purification unit 100 can be shaped and sized to fit within a standard cell of a suspension grid drop ceiling. For instance, the housing 121 of the diffuser air purification unit can have a length of approximately 21 inches, a width of approximately 21 inches, and a height of approximately 12 inches. Similarly, the access panels 124 can have a width of approximately 21 inches and a height of approximately 12 inches.

The diffuser 103 can be a permeable cover placed over an opening at a lower portion of the diffuser air purification unit 100. Air can pass through the diffuser 103 and into the filtration chamber 106. Many different configurations of diffusers can be used with the diffuser air purification unit 100 depending on what is needed or desired. The diffuser 103 can be customized to match any diffusers already present in a setting in which the diffuser purification unit is installed. The diffuser 103 can be selected or customized based upon design preferences of the interior space of a building. Likewise, in some examples the diffuser 103 can be sized and shaped to match standard types of diffusers used in suspension grid drop ceilings. For instance, the diffuser can have a length of approximately 24 inches, a width of approximately 24 inches, and a height of approximately 1-3 inches. This can allow the diffuser air purification unit 100 to blend in with its surroundings, unlike portable air filtration units. In addition, the diffuser 103 can be removed to provide access to internal components of the diffuser air purification unit 100 for maintenance.

The filtration chamber 106 can be a chamber in which air that enters the diffuser air purification unit 100 can be filtered. The filtration chamber 106 can include a lower cavity 127, a filter 130, and an upper cavity 133. The filtration chamber 106 can be located in a portion of the diffuser air purification unit 100 opposite the purification chamber 109.

An upper boundary of the filtration chamber 106 can be defined by an upper surface of the housing 121, and a lower boundary can be defined by the diffuser 103. The filter 130 can be recessed from the diffuser 103 to form the lower cavity 127 of the filtration chamber 106. The lower cavity 127 can allow air that enters the filtration chamber 106 to equalize or disperse along the filter 130 before entering the filter 130. In some examples, the lower cavity 127 can also provide access to the brackets (not shown) used to secure the filter 130 so that the filter 130 can be removed and replaced. Likewise, the filter 130 can be recessed from the upper surface of the housing 121 to form the upper cavity 133 into which air can exit the filter 130 and enter the purification chamber 109. Lateral boundaries of the filtration chamber 106 can be partially defined by the housing 121, the access panels 124, and a separator piece 136. The upper cavity 133 can on one side be open to the purification chamber 109 to allow air to flow from the filtration chamber 106 into the purification chamber 109.

Air can be pulled from the setting in which the diffuser air purification unit 100 is installed, through the diffuser 103, and into the filtration chamber 106 by a blower 139. Air can enter the filtration chamber 106 into the lower cavity 127 and then be pulled upwards through the filter 130. The air can pass through the filter 130 and into the upper cavity 133, and the blower 139 can pull that air into the purification chamber 109.

The filter 130 can filter air from the lower cavity 127 upwards into the upper cavity 133. The filter 130 can be any device designed to remove airborne particulate matter from air or other gasses that flow through the filter 130. The filter 130 can be, for example, a high-efficiency particulate air (HEPA) filter, an electrostatic filter, a washable filter, a fiberglass filter, a pleated filter, or any other suitable air filter. A filter 130 can be chosen based on a Minimum Efficiency Reporting Value (MERV) rating needed in the setting in which the diffuser air purification unit 100 will be used. The filter 130 can also be shaped specially to fit within the filtration chamber 106.

A lower surface of the filter 130 can rest on and be secured to brackets (not shown) located in the lower cavity 127 that are secured to the separator piece 136 and a lateral surface of the housing 121. An upper portion of the filter 130 can contact a gasket 142 that is secured at least to a lateral surface of the housing 121. In some examples, the gasket 142 can also be secured to the separator piece 136, or the gasket 142 and the separator piece 136 can comprise a single piece.

The purification chamber 109 can be a chamber in which air that has been filtered can then be purified. The purification chamber 109 can be located in a portion of the diffuser air purification unit 100 opposite the filtration chamber 106. An upper boundary of the filtration chamber 106 can be defined by an upper surface of the housing 121, and a lower boundary can be defined by a fan deck 145. Lateral boundaries of the filtration chamber 106 can be defined partially by the access panels 124, the separator piece 136, and the housing 121.

The purification chamber 109 can include one or more components suitable for purifying air including, for example, an ultraviolet lamp 148 and a bipolar ionization unit 151. In some examples, however, only one of the ultraviolet lamp 148 and the bipolar ionization unit 151 may be included. In other examples, the purification chamber 109 may include no purification components at all.

The ultraviolet lamp 148 can treat the air that has passed through the filter 130. The ultraviolet lamp 148 can be any device designed to emit ultraviolet radiation capable of inactivating airborne pathogens. For example, the ultraviolet lamp 148 can be a UV-C light. The ultraviolet lamp 148 can be accessed for removal and replacement by removing either of the access panels 124. The ultraviolet lamp 148 can be located in the purification chamber 109 adjacent to an intake of the blower 139. In some examples, the ultraviolet lamp 148 can be secured to a lateral surface of the housing 121 or to the fan deck 145. In some examples, however, the ultraviolet lamp 148 may be omitted, or another purification component may be used in its place.

The bipolar ionization unit 151 can treat the air that has passed through the filter 130. The bipolar ionization unit 151 can be any device designed to produce ions that can cluster around airborne pathogens and cause other airborne particles to clump together. As an example, the bipolar ionization unit 151 can do so by creating and emitting positive and negative oxygen ions without also creating ozone. In some examples, a bipolar ionization unit 151 can be used that requires little-to-no servicing or maintenance. The bipolar ionization unit 151 can be located in the purification chamber 109 and secured to the fan deck 145 or other surface using, for example, a mounting bracket. In some examples, however, the ultraviolet lamp 148 may be omitted, or another purification component may be used in its place.

The purification chamber 109 can further include the blower 139. The blower 139 can pull air from the filtration chamber 106 and into the purification chamber 109. The blower 139 can then pull the air into the blower 139 and expel the air out of the purification chamber 109.

The blower 139 can create pressure that pulls air through the filtration chamber 106 and the purification chamber 109. While in FIGS. 1B, 2B, 3C, 4B, 5B, and 7 the blower 139 is a centrifugal blower, the blower 139 can be any system designed to push and pull air. For example, the blower 139 can be a centrifugal blower, a high-speed blower, an axial fan, a backwards inclined fan, a plug fan, or any other suitable type of blower. The centrifugal blower 139 used can depend on the type of filter 130 used. For example, if the filter 130 is a HEPA filter, a blower 139 can be used that is designed to intake air at a velocity suitable for moving that air through the filter 130.

The blower 139 can pull air into the diffuser air purification unit 100, through filtration chamber 106 and the purification chamber 109, and into the blower 139. A motor can cause an impeller within the blower 139 to spin and pull air into one or more inlets on the sides of the blower 139. The impeller can then expel the air from at outlet on a bottom of the blower 139.

The blower 139, and the one or more inlets in particular, can be located in the purification chamber 109 proximate to the ultraviolet lamp 148, the bipolar ionization unit 151, or other purification components, if any. In some examples, the blower 139 can rest on and be secured to the fan deck 145. The fan deck 145 can include an opening positioned around the outlet of the blower 139 through which air can be expelled from the blower 139. The outlet of the blower 139 can also in some examples include a pair of lip portions that extend downwards through the opening.

In some examples, components included in the purification chamber 109 like the bipolar ionization unit 151 and the blower 139 can be secured to a fan deck 145. The fan deck 145 can be secured to the separator piece 136 and a lateral surface of the housing 121. The fan deck 145 can be removed to allow for maintenance or service of any components secured to the fan deck 145.

The diffuser air purification unit 100 can include access panels 124 that can be removed to provide access to internal components of the diffuser air purification unit 100 for maintenance. For example, either access panel 124 can be removed to provide access to components in the purification chamber 109 like the ultraviolet lamp 148, the bipolar ionization unit 151, and the blower 139. The ultraviolet lamp 148, the bipolar ionization unit 151, or the blower 139 can then be removed individually for maintenance or be replaced altogether. The fan deck 145 can also be removed to allow any components secured to the fan deck 145 to later be maintained or replaced. As another example, either access panel 124 can be removed to provide access to the filtration chamber 106 and the filter 130. The filter 130 can then be removed and replaced. The access panels 124 can be located at the front and back sides of the diffuser air purification unit 100.

The control module 112 can include various components used to control operation of the diffuser air purification unit 100. The control module 112 can be secured to the housing 121 adjacent to the motor of the blower 139. The control module 112 can have, for example, a length of approximately 4 inches, a width of approximately 16.5 inches, and a height of approximately 9 inches.

The occupancy sensor 115 can control operation of the diffuser air purification unit 100 based on the occupancy state of the setting in which the diffuser air purification unit 100 is installed. The occupancy sensor 115 can be a motion sensor, thermal sensor, or other device capable of detecting the presence of a person or other entity within a vicinity of the device. As an example, the occupancy sensor 115 can activate the diffuser air purification unit 100 when one or more persons are detected within the setting in which the diffuser air purification unit 100 is installed. Likewise, the occupancy sensor 115 can, for instance, deactivate the diffuser air purification unit 100 when no person is detected within the setting for a predetermined period of time. In some examples, the occupancy sensor 115 can be secured to an outer portion of the diffuser air purification unit 100.

The status indicator 118 can reflect an operational status of the diffuser air purification unit 100. The status indicator 118 can provide an indication that the diffuser air purification unit 100, for example, is operating normally, requires maintenance, or is not operating. In some implementations, the status indicator 118 can comprise a light-emitting diode or any other light source designed to emit different types or patterns of light in response to different stimuli. The status indicator 118 can indicate that the diffuser air purification unit is operating normally by, for example, emitting a green light or an unblinking light. The status indicator 118 can indicate that the diffuser air purification unit requires maintenance by emitting a yellow light or a blinking light. In some examples, the status indicator 118 can emit different types or patterns of light depending on the type of maintenance needed. For instance, the status indicator 118 can emit a first type or pattern of light when the filter should be changed and a second type or pattern of light when the ultraviolet lamp 148 should be changed. The status indicator 118 can further indicate that the diffuser air purification unit 100 is not operating by emitting no light. In some examples, the occupancy sensor 115 can be secured to an outer portion of diffuser air purification unit 100.

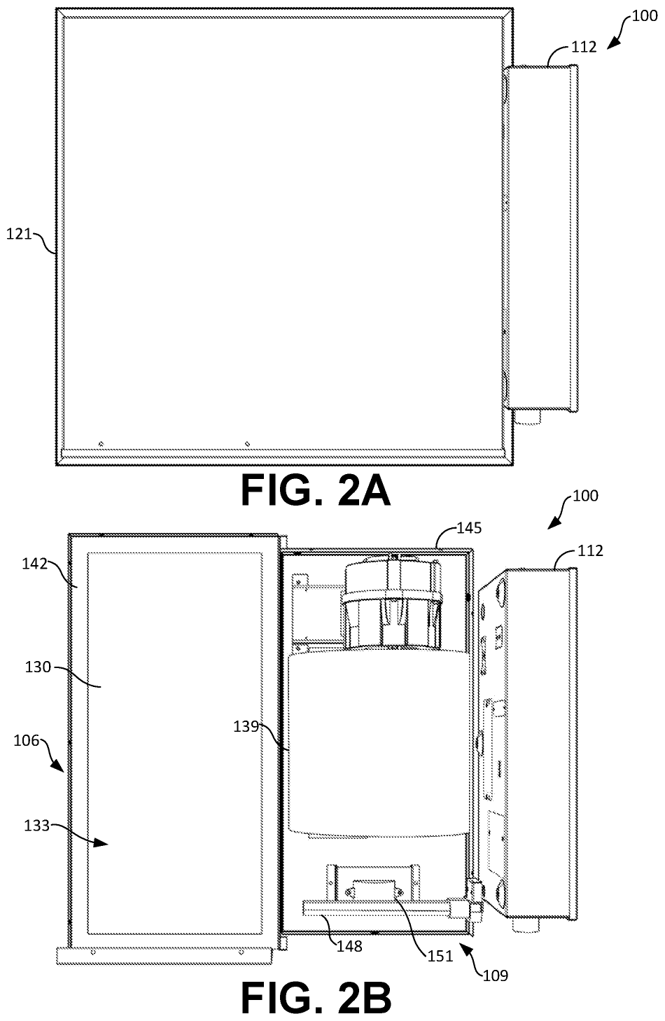

FIGS. 2A-B illustrate examples of top views of the diffuser air purification unit 100. FIG. 2A shows an outside of the diffuser air purification unit 100 from the top view, while FIG. 2B shows internal components of the diffuser air purification unit 100 from the top view. FIG. 2A shows views of the control module 112 and the housing 121. FIG. 2B shows views of the filtration chamber 106, the purification chamber 109, the control module 112, the filter 130, the upper cavity 133, the blower 139, the gasket 142, the fan deck 145, the ultraviolet lamp 148, and the bipolar ionization unit 151.

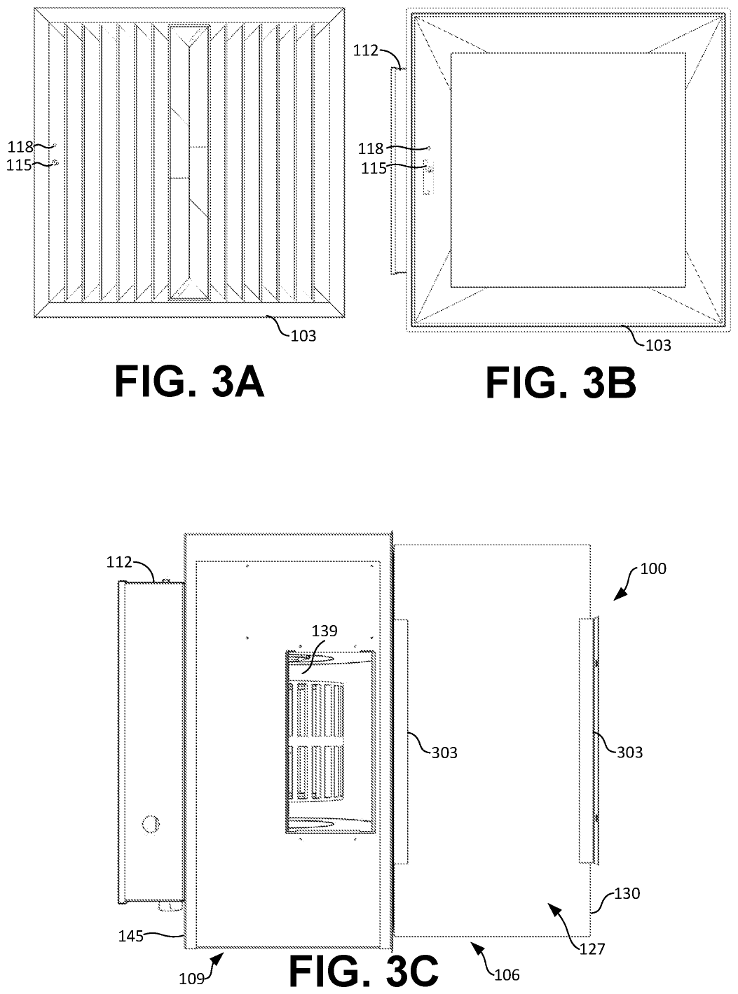

FIGS. 3A-C illustrate examples of bottom views of the diffuser air purification unit 100. FIGS. 3A and 3B show an outside of the diffuser air purification unit 100 from the bottom view, while FIG. 3C shows internal components of the diffuser air purification unit 100 from the bottom view. FIG. 3A shows views of the diffuser 103, the occupancy sensor 115, and the status indicator 118. FIG. 3B shows views of an alternative diffuser 103, the control module 112, the occupancy sensor 115, and the status indicator 118. FIG. 3C shows views of the filtration chamber 106, the purification chamber 109, the control module 112, the lower cavity 127, the filter 130, the blower 139, the fan deck 145, and brackets 303. A lower surface of the filter 130 can rest on the brackets 303 located in the lower cavity 127 and secured to the separator piece 136 and a lateral surface of the housing 121. The lower cavity 127 can also provide access to the brackets 303 so that the filter 130 can be removed and replaced. In some examples, the brackets 303 can be secured using hand bolts to allow for easy removal and replacement of the filter 130.

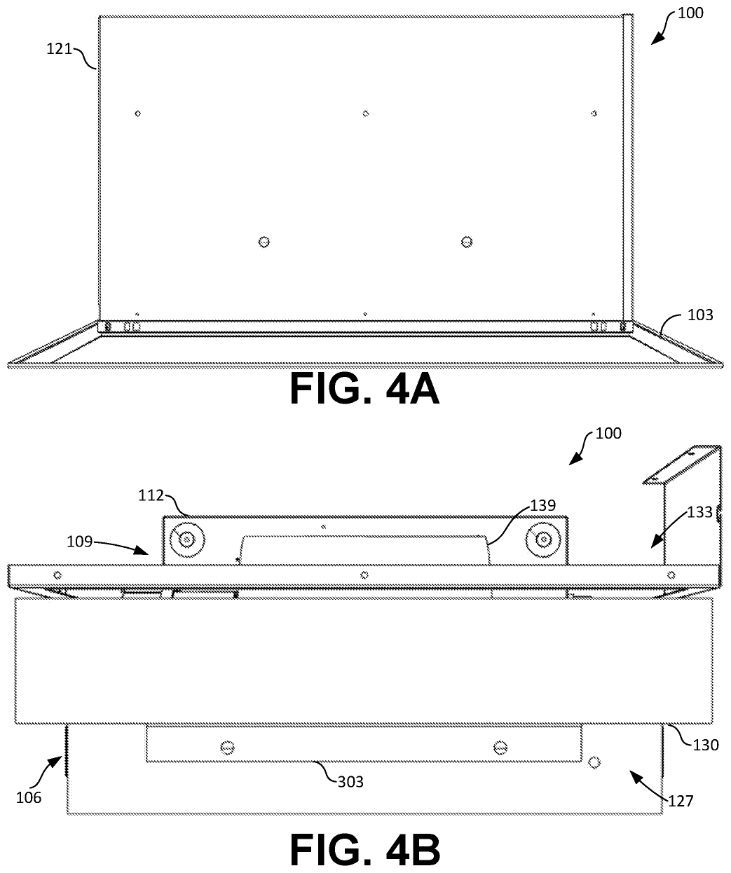

FIGS. 4A-B illustrate examples of first side views of the diffuser air purification unit 100. FIG. 4A shows an outside of the diffuser air purification unit 100 from the first side view, while FIG. 4B shows internal components of the diffuser air purification unit 100 from the first side view. FIG. 4A shows views of the diffuser 103 and the housing 121. FIG. 4B shows views of the filtration chamber 106, the purification chamber 109, the control module 112, the lower cavity 127, the filter 130, the upper cavity 133, the blower 139, and a bracket 303.

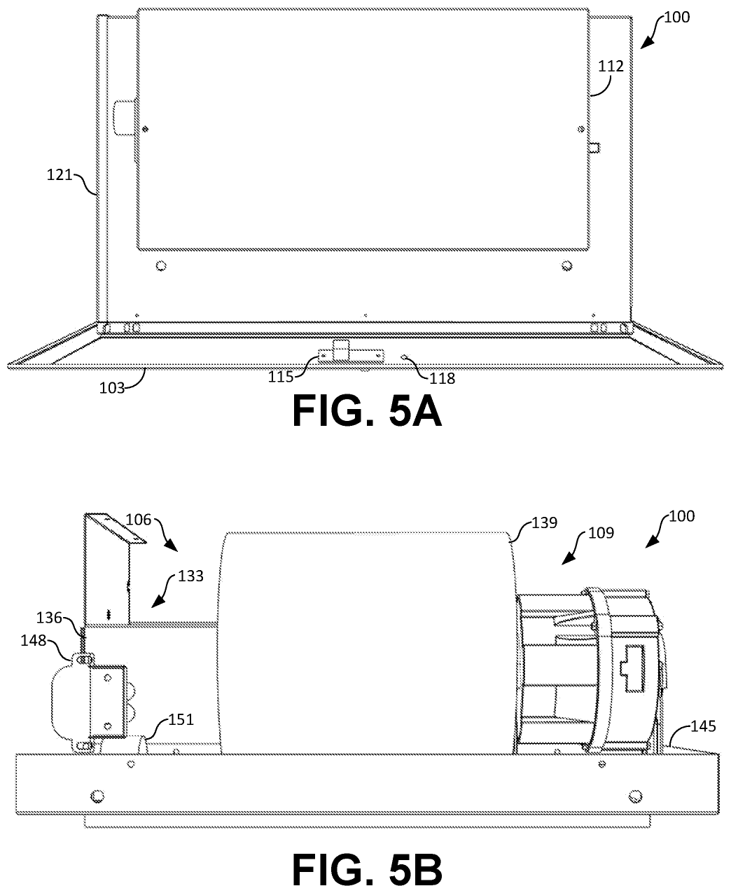

FIGS. 5A-B illustrate examples of second side views of the diffuser air purification unit 100. FIG. 5A shows an outside of the diffuser air purification unit 100 from the second side view, while FIG. 5B shows the internal components of the diffuser air purification unit 100 from the second side view. FIG. 5A shows the diffuser, the control module 112, the occupancy sensor 115, the status indicator 118, and the housing 121. FIG. 5B shows the filtration chamber 106, the purification chamber 109, the separator piece 136, the blower 139, the fan deck 145, the ultraviolet lamp 148, and the bipolar ionization unit 151.

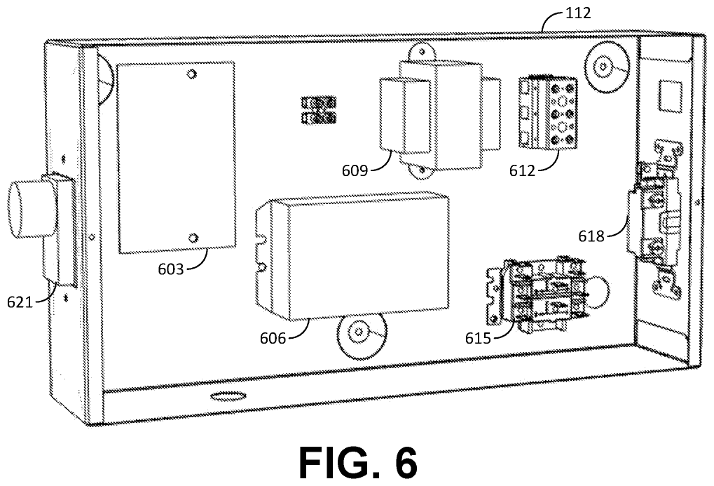

FIG. 6 illustrates an example of an isolated perspective view of a control module 112 of the diffuser air purification unit 100. The control module 112 can include a control unit 603, an ultraviolet ballast 606, a transformer 609, a terminal block 612, a fan relay 615, a disconnect switch 618, and a speed controller 621. The control unit 603, ultraviolet ballast 606, transformer 609, terminal block 612, and fan relay 615 can be secured to an inside surface of the control module 112. The control unit 603 can be any microcontroller, circuit board, or other electronic device configured control operation of the various functions of the diffuser air purification unit 100. The ultraviolet ballast 606 can be any electronic, magnetic, or electro-mechanical device designed to provide voltage sufficient to initiate the emission of ultraviolet light from the ultraviolet lamp 148 and to sustain the emission of the ultraviolet light during operation of the ultraviolet lamp 148. The transformer 609 can be any electrical device designed to supply voltage used to operate the motor of the blower 139. The terminal block 612 can be any device designed to secure wires or any other connections used to operate the motor of the blower 139. The fan relay 615 can be any electrical device designed to relay power used to operate the motor of the blower 139.

Further, the disconnect switch 618 and the speed controller 621 can each be secured partly to an inner and partly to an outer surface of the control module 112. The disconnect switch 618 can be any electrical device designed to cut power used to operate the motor of the centrifugal motor. The speed controller 621 can be any device used to control a speed at which the motor of the blower 139 operates and therefore to control the speed of airflow in and out of the diffuser air purification unit 100. In some examples, the speed controller 621 can allow for selection of one or more set speeds. In other examples, the speed controller 621 can allow for selection of a variable speed. The speed controller 621 can in some examples be controlled by a computing device such that the computing device can control the airflow speed of the diffuser air purification unit 100. The speed controller 621 can be automated to set an airflow speed of the diffuser air purification unit 100 based on conditions like a size of the setting in which the diffuser air purification unit 100 is installed.

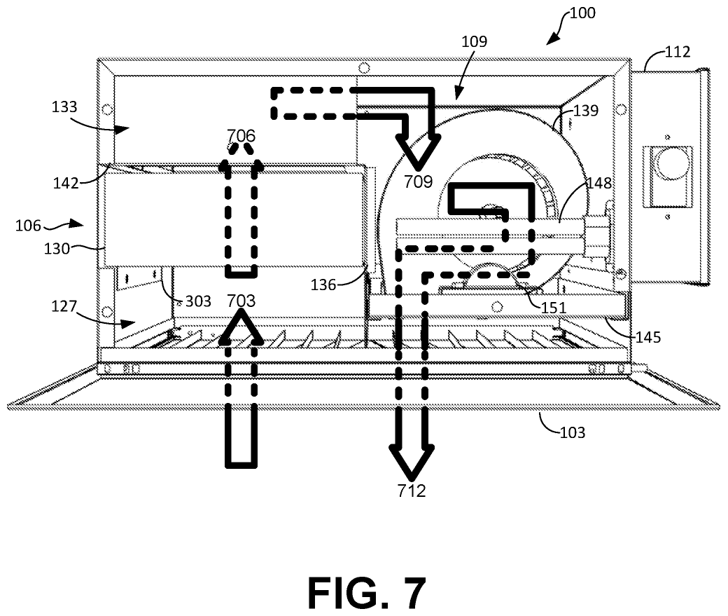

FIG. 7 illustrates an example of a flow diagram of a process for purifying air using the diffuser air purification unit 100. To illustrate this process a front view of the diffuser air purification unit 100 is shown with an access panel 124 removed.

At step 703, air can enter the filtration chamber 106 through the diffuser 103. Air can be pulled upwards into the lower cavity 127 of the filtration chamber 106 by the blower 139. The air can enter the filtration chamber 106 from a room or other setting in which the diffuser air purification unit 100 is installed.

At step 706, the air can pass through filter 130. The blower 139 can pull air from the lower cavity 127 and forces air through the filter 130. Because of the pressure created by the blower 139 and the air's enclosure in the lower cavity 127 of the filtration chamber 106, the air is forced through the filter 130. The filter 130 can filter airborne particles from the air. Once the air passes through the filter 130, the air can enter the upper cavity 133 of the filtration chamber 106.

At step 709, the air can enter the purification chamber 109 from the filtration chamber 106. The blower 139 can pull air from the upper cavity 133 of the filtration chamber 106 into the purification chamber 109. Any purification components inside the purification chamber 109 like the ultraviolet lamp 148 or the bipolar ionization unit 151 can purify the air that flows through the purification chamber 109.

At step 712, after being purified, air within the purification chamber 109 can enter the blower 139 and be expelled from the blower 139 into the setting in which the diffuser air purification unit 100 is installed. The impeller can pull air into the blower 139 through the one or more inlets located on the sides of the blower 139. The impeller can then expel the air from the blower 139 through the outlet located on a bottom portion of the blower 139. The outlet can extend through an opening in the fan deck 145. The air expelled from the outlet can pass through the diffuser 103 and into the setting.

It should be noted that measurements, amounts, and other numerical data can be expressed herein in a range format. It is also understood that there are a number of values disclosed herein, and that each value is also herein disclosed as “approximately” that particular value in addition to the value itself. For example, if the value “10” is disclosed, then “approximately 10” is also disclosed. Similarly, when values are expressed as approximations, by use of the antecedent “approximately,” it will be understood that the particular value forms a further aspect. For example, if the value “approximately 10” is disclosed, then “10” is also disclosed.

As used herein, the terms “about,” “approximately,” “at or about,” and “substantially equal” can mean that the amount or value in question can be the exact value or a value that provides equivalent results or effects as recited in the claims or taught herein. That is, it is understood that amounts, sizes, measurements, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In general, an amount, size, measurement, parameter or other quantity or characteristic is “about,” “approximate,” “at or about,” or “substantially equal” whether or not expressly stated to be such. It is understood that where “about,” “approximately,” “at or about,” or “substantially equal” is used before a quantitative value, the parameter also includes the specific quantitative value itself, unless specifically stated otherwise.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y’, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y’, and ‘greater than z’. In addition, the phrase “about ‘x’ to ‘y’”, where ‘x’ and ‘y’ are numerical values, includes “about ‘x’ to about ‘y’”.

Such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

A phrase, such as “at least one of X, Y, or Z,” unless specifically stated otherwise, is to be understood with the context as used in general to present that an item, term, etc., can be either X, Y, or Z, or any combination thereof (e.g., X, Y, and/or Z). Similarly, “at least one of X, Y, and Z,” unless specifically stated otherwise, is to be understood to present that an item, term, etc., can be either X, Y, and Z, or any combination thereof (e.g., X, Y, and/or Z). Thus, as used herein, such phrases are not generally intended to, and should not, imply that certain embodiments require at least one of either X, Y, or Z to be present, but not, for example, one X and one Y. Further, such phrases should not imply that certain embodiments require each of at least one of X, at least one of Y, and at least one of Z to be present.

Although embodiments have been described herein in detail, the descriptions are by way of example. The features of the embodiments described herein are representative and, in alternative embodiments, certain features and elements may be added or omitted. Additionally, modifications to aspects of the embodiments described herein may be made by those skilled in the art without departing from the spirit and scope of the present disclosure defined in the following claims, the scope of which are to be accorded the broadest interpretation so as to encompass modifications and equivalent structures.

Therefore, at least the following is claimed:

1. A diffuser air purification unit comprising:
a diffuser;
a filtration chamber configured to receive air through the diffuser from an external setting in which the diffuser air purification unit is installed and to cause a filtration of the air when the air flows through the filtration chamber;
a purification chamber configured to receive the air from the filtration chamber and to cause a purification of the air when the air flows through the purification chamber;
a blower configured to intake the air from the external setting through the diffuser, the filtration chamber, and the purification chamber and to expel the air into the external setting;
a housing at least partially enclosing the filtration chamber and the purification chamber; and
a control module secured to the housing, the control module being configured to control an operation of the diffuser air purification unit.

2. The diffuser air purification unit of claim 1, wherein the diffuser comprises a permeable cover secured to a lower portion of the housing.

3. The diffuser air purification unit of claim 2, wherein the diffuser is removable from the housing.

4. The diffuser air purification unit of claim 1, further comprising an occupancy sensor secured to the diffuser, the occupancy sensor being configured to activate the diffuser air purification unit in response to detecting an occupant in the external setting and to deactivate the diffuser air purification unit in response to failing to detect an occupant in the external setting following a predetermined period of time.

5. The diffuser air purification unit of claim 1, further comprising a status indicator secured to the diffuser, the status indicator configured to provide an indication of a status of the diffuser air purification unit.

6. The diffuser air purification unit of claim 1, wherein the filtration chamber comprises a lower cavity, a filter, and an upper cavity, wherein the filtration of the air that flows through the filtration chamber is caused when the air flows from the lower cavity, through the filter, and into the upper cavity.

7. The diffuser air purification unit of claim 1, wherein the purification chamber comprises an ultraviolet lamp and a bipolar ionization unit, wherein the purification of the air that flows through the purification chamber is caused by the ultraviolet lamp.

8. The diffuser air purification unit of claim 1, wherein the purification chamber comprises an ultraviolet lamp, wherein the purification of the air that flows through the purification chamber is caused by the ultraviolet lamp.

9. The diffuser air purification unit of claim 1, wherein the purification chamber comprises a bipolar ionization unit, wherein the purification of the air that flows through the purification chamber is caused by the bipolar ionization unit.

10. The diffuser air purification unit of claim 1, wherein the blower comprises a centrifugal blower, a high-speed blower, an axial fan, a backwards inclined fan, or a plug fan.

11. An apparatus, comprising:
a diffuser;
a filter configured to cause a filtration of air received through the diffuser from an external setting;
an ultraviolet lamp configured to cause a treatment of the air; and
a blower configured to intake the air from the external setting through the diffuser and to expel the air into the external setting through the diffuser following the filtration and the treatment.

12. The apparatus of claim 11, further comprising a bipolar ionization unit configured to produce ions that mix with the air.

13. The apparatus of claim 12, wherein the bipolar ionization unit is located proximate to the blower.

14. The apparatus of claim 11, further comprising a housing at least partially enclosing the filter, the ultraviolet lamp, and the blower.

15. The apparatus of claim 14, wherein the diffuser comprises a permeable cover secured to a lower portion of the housing.

16. The apparatus of claim 15, wherein the diffuser is removable from the housing.

17. The apparatus of claim 11, further comprising a control module configured to control an operation of the blower.

18. The apparatus of claim 11, further comprising an occupancy sensor, the occupancy sensor being configured to cause an activation of the blower in response to detecting an occupant in the external setting and to cause a deactivation of the blower in response to failing to detect an occupant in the external setting following a predetermined period of time.

19. The apparatus of claim 11, wherein the blower comprises a centrifugal blower, a high-speed blower, an axial fan, a backwards inclined fan, or a plug fan.

20. The apparatus of claim 11, wherein the filter comprises a high efficiency particulate air (HEPA) filter.
